(19) United States
(12) Patent Application Publication
(10) Pub. No.: US 2009/0232884 A1
(43) Pub. Date: Sep. 17, 2009

(54) Composition and method for treatment of the side-effects associated with administration of cancer chemotherapeutic agents

(76) Inventor: John P. Ford, Unadilla, NY (US)

Correspondence Address: STRASBURGER & PRICE, LLP, 901 MAIN STREET, SUITE 4400, DALLAS, TX, 75202, US

(21) Appl. No.: 12/381,474

(22) Filed: Mar. 12, 2009

Related U.S. Application Data
(60) Provisional application No. 61/069,031, filed on Mar. 12, 2008.

(51) Int. Cl. A61K 9/127

(52) U.S. Cl. 424/450

(57) ABSTRACT

A composition and method for the treatment of the side-effects associated with the administration of cancer chemotherapeutic agents involves the oral ingestion of cationic liposomes containing the substrates orotate, UMP and AMP. The cationic liposomes bind to the cells lining the mucosa of the intestinal tract and then the contents of the cationic liposome are then taken up in the interior of the cells to prevent the metabolism or anabolism of the cancer treatment drug 5-FU into a toxic species.

### CROSS REFERENCE TO RELATED APPLICATION

This application claims the benefit of Provisional U.S. patent application Ser. No. 61/069,031 filed Mar. 12, 2008.

### STATEMENT REGARDING FEDERALLY FUNDED RESEARCH AND DEVELOPMENT

The invention described in this patent application was not the subject of federally sponsored research or development.

### FIELD

The present invention pertains to a composition and method for ameliorating the effect of drugs on the human body; more particularly, the present invention pertains to a composition and method for treatment of the side-effects associated with the administration of cancer chemotherapeutic agents.

### BACKGROUND

Human beings who develop cancer are often treated with chemotherapeutic drugs. Cancer chemotherapeutic drugs, while effective at destroying a cancerous tumor, may also cause damage to normal tissues of the body. The normal tissues of the body most often affected by the side-effects of a cancer chemotherapeutic drug include the lining of the mouth, the lining of the intestine and the hair. Symptoms associated with the deleterious effects of chemotherapeutic cancer drugs include hair loss, nausea and vomiting. Occasionally, the side-effects associated with the administration of cancer chemotherapeutic drugs can be debilitating and result in interruptions of the cancer chemotherapeutic drug treatment regimen.

Various attempts have been made to lessen or to eliminate the symptoms associated with the administration of cancer chemotherapeutic drugs. Other drugs, such as steroids, have been administered to patients to alleviate the suffering associated with the side-effects of cancer treatment using chemotherapy. The success associated with the use of these other drugs to alleviate suffering is problematic.

One of the problems associated with the use of other drugs to alleviate the suffering associated with the side-effects of cancer treatment using chemotherapy is delivering the mitigating drug directly to the organ affected by the cancer treatment drug causes undesirable organ toxicity.

Another problem associated with these other drugs is that drugs such as steroids and other drugs used to alleviate the side-effects of cancer drugs may be toxic to other tissues. Such tissue toxicity represents additional unwanted side-effects.

A third problem associated with drugs administered to alleviate the side-effects of cancer therapy is that the drug used to alleviate the side-effects caused by the cancer drug may interfere with the activity of the cancer drug resulting in diminished effectiveness for destroying the targeted cancerous tumor.

Accordingly, there remains a need in the art for a composition and method for the treatment of the side-effects associated with the administration of cancer chemotherapeutic agents.

### SUMMARY

A composition and method for the treatment of the side-effects associated with administration of cancer chemotherapeutic agents is disclosed herein.

According to the present invention cationic liposomes containing the substrates orotic acid (orotate), uridylic acid (UMP), and adenosine monophosphate (AMP) are administered orally to patients receiving 5-FU for cancer treatment. The cationic liposomes containing the substrates orotate, UMP and AMP bind to the cells lining the mucosa of the intestinal tract. After binding to the cells lining the mucosa of the intestinal tract, the substrate contents of the cationic liposome (orotate, UMP and AMP) are deposited into the interior of the cells lining the mucosa of the intestinal tract. The substrates orotate and UMP prevent the metabolism of 5-FU into a toxic species, thereby protecting those cells into which the cationic liposome has been deposited from the toxicity associated with administration of 5-FU. The substrate AMP protects the cells lining the mucosa of the intestinal tract from any toxicity associated with the administration of the substrate orotate.

Because the cells lining the mucosa of the intestinal tract are protected from the toxic effects of 5-FU by the cationic liposomes containing the substrates orotate, UMP and AMP, the dosage of 5-FU or its active precursor may be increased so as to more effectively destroy the existing cancerous tumor. Moreover, since 5-FU is not being metabolized in the gastrointestinal tract, more 5-FU is available for destruction of the targeted cancerous tumor. Since the toxic side-effects associated with 5-FU metabolism have been ameliorated by the composition and method of the invention disclosed herein, normal homeostasis is maintained in the GI tract and patients receiving 5-FU for cancer therapy are more likely to adhere to the treatment regimen recommended by their physician. The combination of increased compliance and increased dosage of 5-FU afforded by the invention disclosed herein improves the outcome associated with 5-FU cancer treatment therapy.

### BRIEF DESCRIPTION OF THE DRAWING FIGURES

A still better understanding of the composition and method for treatment of the side-effects associated with the administration of cancer chemotherapeutic agents may be had by reference to the following drawing figure which is used to illustrate the following DESCRIPTION OF THE EMBODIMENTS wherein:

FIG. 1 is a schematic description of a cationic head group attached to a hydrophobic group via a linker.

### DESCRIPTION OF THE EMBODIMENTS

The composition and method of treating the side-effects of cancer chemotherapeutic drug administration disclosed herein employs substrates of the enzymes which the human body uses to metabolize the cancer treatment drug. The substrates of the enzymes are incorporated into cationic liposomes. Such metabolic by-products and enzyme substrates are incorporated into the cationic liposomes to prevent the damage caused by the cancer drug. Oral administration of the cationic liposome promotes incorporation of the enzyme substrates into the cells lining the mucosa of the intestinal tract to which the cancer drug is toxic. Thereby the toxic effect of the cancer drug is minimized. These substrates of the enzymes used to metabolize the cancer treatment drug are nontoxic to normal tissues. By administering the substrates of the enzymes which metabolize the cancer chemotherapeutic drug by orally administered cationic liposomes, the substrates of the enzymes used to metabolize the cancer treatment drug are delivered only to the cells lining the mucosa of the intestinal tract and do not interfere with the activity of the cancer chemotherapeutic drug on the cancerous tumor to which the cancer chemotherapeutic drug is directed.

The invention disclosed herein is specifically directed to a composition and method for alleviating the gastrointestinal side-effects of 5-fluorouracil (5-FU) but may, in general, be applicable to alleviating the side-effects of other cancer chemotherapeutic drugs which cause GI toxicity. The cancer chemotherapeutic agent, 5-FU is one of the first cancer drugs identified and is commonly used in the treatment of cancers of the colon, breast, stomach and other organs of the body. Among other side effects, the common side-effects of 5-FU administration are nausea and diarrhea because of the toxicity exerted by 5-FU on the cells lining the mucosa of the intestinal tract. A chart of the metabolic pathway for 5-FU appears at FIG. 2 on page 6 of 11 of Indigo Biosciences, Inc. report R 081126-1 dated Jul. 9, 2006 by J. Vanden Heuvel and which is incorporated herein by reference.

The chemical 5-FU is inactive against cancerous tumors until it is metabolized by the body to the active species. Anabolism of 5-FU to its active species is accomplished by one of three alternate enzyme systems which are as follows: 1) uridine phosphorylase (UP), 2) thymidine phosphorylase (TP) and 3) orotate phosphoribosyl transferase (OPT). There is evidence that metabolism of 5-FU by OPT results in the metabolite which is toxic to the gastrointestinal tract.

Enzymes such as UP, TP and OPT can be blocked from acting on the primary substrate (e.g. 5-FU) by saturating the active site of the enzyme with a non-competiting substrate. Saturation of the active site of an enzyme with a competing substrate decreases the primary substrate of the enzyme from entering the active site of the enzyme where it is metabolized by the enzyme. If the primary substrate has decreased the active access to the enzyme, the primary substrate is metabolized less by the enzyme.

Orotate is the natural substrate of the enzyme OPT. The activity of the enzyme OPT on 5-FU will be decreased by the presence of an excess by saturating the active sites on the enzyme OPT by the natural substrate orotate. The production of toxic metabolite of 5-FU will be reduced by the metabolism of 5-FU by the enzyme OPT because the active site of the enzyme OPT is filled with the competing substrate orotate. The competing substrate orotate decreases 5-FU from entering the active site of the enzyme OPT where it can be further metabolized to the toxic product. Attempts to use the substrate orotate to lessen the toxic effects of 5-FU on the GI tract have previously met with failure because the competing substrate orotate is not taken up efficiently by the cells lining the mucosa of the intestinal tract. The competing substrate orotate does not readily cross the cell membrane of the cells lining the mucosa of the intestinal tract because the substrate orotate has an acidic pK and an overall negative charge as do the cell membranes of the cells lining the mucosa of the intestinal tract. Since like negative charges repel, the substrate orotate cannot cross the cell membrane. Accumulation of 5-FU by cells lining the mucosa of the intestinal tract occurs readily because 5-FU is near its pK of 8 when administered to humans.

The invention disclosed herein is a method and composition to promote accumulation of the substrate orotate in the cells lining the mucosa of the intestinal trace by incorporating the substrate orotate with certain substrates of the enzymes into cationic liposomes. Cationic liposomes carry a net positive charge. This net positive charge enables the cationic liposome to be taken up by the negatively charged outer membranes of the cells lining the mucosa of the intestinal tract. By incorporating the substrate orotate and other substrates of enzymes into cationic liposomes, the substrate orotate and the other substrates of enzymes can be delivered to the cells lining the mucosa of the intestinal tract and then be taken up by the individual cells of the intestinal tract. Once taken up by the individual cells of the intestinal tract, the competing substrate orotate can then reduce the activity of the enzyme OPT in metabolizing 5-FU to its toxic by-product by saturating the active site of the enzyme OPT. This saturation of the active site of the enzyme OPT decreases further metabolism of 5-FU by the enzyme OPT. The cells lining the mucosa of the intestinal tract are thus relatively protected from the toxic by-products of 5-FU metabolism. UMP and AMP included with the substrate orotate in the cationic liposome further decreases the metabolism of 5-FU as well as mitigating toxicity from the administration of the substrate orotate. The cationic liposomes, because of their charge, will minimally permeate the body and release the substrate orotate to alter 5-FU metabolism in the cancerous tumor.

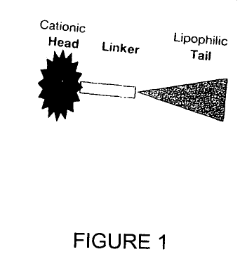

Many cationic lipids have been developed and used in cationic liposome preparation. While the cationic lipids have many different chemical structures, all the cationic lipids are all composed of a cationic head group composed of primary, secondary, tertiary or quatemary amines. The primary, secondary, or quatemary amines are attached to a hydrophobic group via a linker as shown in FIG. 1.

When placed in an aqueous solution, the cationic lipids form liposomes in which the cationic head is on the outside surface of the liposome and the hydrophobic group is on the inside of the liposome. By use of this process a three dimensional bag or sack is formed. The three-dimensional bag or sack contains the substrate orotate and other substrates of enzymes within the interior of the cationic liposome. Since the cationic head of the lipid is located on the surface of the cationic liposome, the positive charge associated with the cationic lipid is located on the outside surface of the cationic liposome.

Some of the commercially available cationic lipids and their suppliers are listed below:
Lipofect ACE (Life Technologies)
Lipofection (Life Technologies)
LipofectAmine (Life Technologies)
CeliFectin (Life Technologies)
DMRIE-C (Life Technologies)
DDAB (Sigma)
DC-Chol (Sigma)
DOTAP (Boehringer Mannheim, Avanti Polar Lipids, Biontex)
MRX-230 and MRX-220 (Avanti Polar Lipids)
Transfectam (Promega)
Transfast (Promega)
Tfx 10, Tfx 20, and Tfx 50 (Promega)
Prefection-CaPO4 (Promega)
Prefection-DEAE-Dextran (Promega)
GeneSHUTTLE-40 (Quantum BiotechnoIogies)
CLONfectin (Clontech)
METAFECTENE (Biontex)
INSECTOGENE (Biontex)
Effectene (Qiagen)
FuGENE 6 (Roche Molecular Biochemicals)
GENESEAL (MTTI)

In preparing the cationic liposome, a cationic lipid or a combination of cationic lipids is/are dissolved in an organic solvent such as chloroform or methanol. The solvent is then removed by use of a vacuum or by blowing an inert gas over the solution followed by rehydration in an aqueous solution. The aqueous solution used for rehydration contains the substrate orotate and other substrates of enzymes which metabolize the cancer treatment drug. As the cationic liposome forms due to hydrophobicity of the cationic lipids, the aqueous solution containing the substrate orotate and other substrates of enzymes which metabolize the cancer treatment drug are trapped within the interior of the cationic liposome. The cationic liposomes containing the substrate orotate and other substrates of enzymes which metabolize the cancer treatment drug are then made to a uniform size by either sonication or membrane extrusion.

Additionally, it is disclosed that, in addition to the substrate orotate, it is necessary to incorporate into the aqueous contents of the cationic liposome other substrates of enzymes which metabolize the cancer treatment drug to further prevent metabolism of 5-FU and mitigate toxicity which is caused by the competing substrate orotate. As mentioned above, 5-FU is also, alternatively, metabolized to its toxic form by the two enzymes UP and TP. Uridylic acid (UMP) is dephosphorylated in the cell by various acid and akaline phosphates, including 5′ neucliotidose, to uridine which is a subtrate for UP. UP converts uridine to uracil. In the reverse reaction, uracil can then non-competitively compete for anabolism with 5-FU via TP and UP to decrease cell toxicity. Intracellularly UMP is degraded to uracil. In conditions of excess uracil, the active site on the UP and TP enzymes will be filled with uracil thereby decreasing in metabolism of 5-FU by enzymes UP and TP to the toxic form. Incorporation of UMP into the cationic liposome and delivery by the cationic liposome of UMP to the cells of the gastrointestinal tract prevents metabolism of 5-FU in the cells lining the mucosa of the gastrointestinal tract and thereby prevents production of the toxic anabolic of 5-FU, FDUMP(Fluorour Deoxy Uridine Monophosphate). The combined use of the substrate orotate and UMP decreases the anabolism of 5-FU all three enzyme systems (UP, TP, and OPT) involved in the metabolism of 5-FU.

The competing substrate dietary orotate is probably nontoxic; as it permeates the cell membrane poorly because of its electrical charge. However, it has been observed that cellular exposure to enough orotate such that the orotate permeates the cell memebrane depletes phosphosho ribosyl pyrophosphate (PRPP) which is linked to the substrate orotate by the enzyme OPT to form a monophosphate pyrimidine precursor. As purine de novo synthesis requires depletion of PRPP by administration of the substrate orotate can cause cell death by decreasing purine synthesis and thus can cause “purine less death”. This cellular growth imbalance and tumor formation may be neutralized by the addition of adenosine monophosphate (AMP). AMP is, in part, converted to inosine monophosphate(IMP). IMP is a precursor to GMP. Thus purine precursors AMP and GMP are available to the cells neutralizing toxicity. The gastro-intestinal mucosa, not the tumor, is protected from the toxicity of 5-FU. Thus, it is further disclosed herein that AMP may be included with the substrate orotate and UMP within the aqueous interior of the cationic liposome. The use of changed species decreases the likelihood of “salvaging” tumor cells from the intended toxicity of 5-FU. The cationic liposome is a targeted vector to gastro-intestinal mucosa and does not provide a systemic source of neucleotide precursors.

Those of ordinary skill in the art will understand that the present invention discloses the use of liposomes to deliver a substance such as the substrates orotate, UMP and/or AMP to the cells lining the mucosa of the intestinal tract without raising the systemic levels of the delivered material. The reason for not raising the systemic levels of the delivered material is that a substance such as the substrate orotate may salvage a tumor from the effects of the 5-FU chemotherapy. Liposomes have never been used to accomplish this function. Rather, liposomes are typically used to cause a substance to enter a cancerous tumor or to enter into an organ such as the liver. Herein, a substance like the substrate orotate is used only to get into the cells lining the mucosa of the intestinal tract. Such function then becomes an asymmetric drug delivery system and method wherein different amounts of a drug are delivered to different parts of the body.

While the composition and method of the present invention have been disclosed according to their preferred and alternate embodiments, those of ordinary skill in the art once having lead to foregoing disclosure. Such other embodiments shall be included within the scope and meaning of the appended claims.

I claim:

1. A composition for the treatment of gastrointestinal toxicity caused by administration of cancer chemotherapeutic agents to cancer patients, said composition comprising:
cationic liposomes;
an aqueous solution inside said cationic liposomes, said aqueous solution containing metabolic by-products and substrates of enzymes which metabolize or anabolize the cancer chemotherapeutic agents which metabolic by-products and substrates inhibit metabolism of the cancer chemotherapeutic agent to a toxic species but which metabolic by-products and substrates are non-toxic wherein normal homeostasis is maintained in the GI tract.

2. The composition as defined in claim 1 wherein the aqueous solution further includes uridylic acid.

3. The composition as defined in claim 2 further including adenosine monophosphate.

4. A composition for the treatment of the gastrointestinal toxicity caused by administration of 5-fluorouracil to cancer patients, said composition comprising:
cationic liposomes;
an aqueous solution inside said cationic liposomes, which aqueous solution includes the substrates orotate, UMP and AMP.

5. The composition as defined in claim 4 further including uridylic acid.

6. The composition as defined in claim 2 further including adenosine monophosphate.

7. A method for treatment of the gastrointestinal toxicity caused by administration of cancer chemotherapeutic agents to cancer patients, said method comprising the steps of:
preparing cationic liposomes containing metabolic by-products and substrates of enzymes which metabolize the cancer therapeutic agents to inhibit the metabolism of the cancer chemotherapeutic agent to a toxic species;
administering said cationic liposomes containing metabolic by-products and said substrates by oral ingestion to the cancer patient to inhibit the metabolism of the cancer chemotherapeutic agent to a toxic species in patients receiving the cancer chemotherapeutic agent.

8. The method defined in claim 7 further including the step of adding uridylic acid to said cationic liposomes.

9. The method as defined in claim 8 further including the step of adding adenosine monophosphate to said cationic liposomes.

10. A method for the treatment of gastrointestinal toxicity caused by the administration of 5-fluorouracil to cancer patients, said method comprising the steps of:
preparing cationic liposomes containing the substrates orotate, UMP and AMP;
administering said cationic liposomes containing the substrates orotate, UMP and AMP to patients receiving 5-fluorouracil for cancer therapy by oral ingestion.

11. The method defined in claim 10 further including the step of adding uridylic acid to said cationic liposomes.

12. The method as defined in claim 11 further including the step of adding adenosine monophosphate.

13. A method for preparing a cationic liposome, said method comprising the steps of:
dissolving a cationic lipid in an organic solvent;
removing the solvent;
rehydrating said cationic lipid in an aqueous solution wherein substrates of enzymes which metabolize a cancer treatment drug are trapped within the interior of the cationic lipid forming the cationic lipid into a uniform size.
